(19) United States
(12) Patent Application Publication
(10) Pub. No.: US 2022/0095899 A1
(43) Pub. Date: Mar. 31, 2022

(54) EXPANDABLE GUIDE DEVICES, SYSTEMS, AND METHODS

(71) Applicant: Boston Scientific Scimed, Inc., Maple Grove, MN (US)

(72) Inventors: Brittany Elizabeth Reed, Brighton, MA (US); Hannah Rebar, Brighton, MA (US); Judith Beaudoin Frontin, Boston, MA (US); James Weldon, Newton, MA (US); Michael Anthony Melito, South Amboy, NJ (US); Aram Lee, Boston, MA (US); Max A. Berman, Eliot, ME (US); Paul Smith, Smithfield, RI (US); Conor Reid, Cambridge, MA (US); Jan Weber, Maastricht (NL); Andrea Cahill, Cambridge, MA (US); Tara Ann Jarobski, North Oxford, MA (US); Tyler Cloyd, Boston, MA (US); Nestor Allan Ibanez, Brighton, MA (US)

(21) Appl. No.: 17/476,976

(22) Filed: Sep. 16, 2021

Related U.S. Application Data
(60) Provisional application No. 63/083,962, filed on Sep. 27, 2020.

(51) Int. Cl. A61B 1/01; A61B 1/31; A61B 1/005; A61B 1/018

(52) U.S. Cl. CPC A61B 1/01; A61B 1/018; A61B 1/0058; A61B 1/31

(57) ABSTRACT

The present disclosure, in its various aspects, is directed to expandable guide devices, implementation methods, and related systems. In one example, an expandable guide device is configured to receive an instrument through an instrument lumen of the device. Each of a plurality of expandable members are disposed circumferentially about a body of the device that defines the instrument lumen, and each expandable member is configured to be independently transitionable between an expanded configuration and an unexpanded configuration.

### PRIORITY

The present application is a non-provisional of and claims the benefit of priority under 35 USC § 119 to, U.S. Provisional Application Ser. No. 63/083,962, filed Sep. 27, 2020, the disclosure of which is herein incorporated herein by reference in its entirety and for all purposes.

### FIELD

The present disclosure pertains to medical devices. More particularly, the present disclosure pertains to expandable guide devices and related systems and methods, particularly as may increase the effectiveness and efficiency of endoscopic procedures.

### BACKGROUND

Endoscopes, for example, are used in medical procedures to examine and treat conditions within the digestive tract. ERCP procedures, for example, are used to examine, diagnose and treat areas in the common bile duct and pancreatic ducts. In some procedures, cannulation of the bile duct can be difficult, with movement in the duodenum making positioning and stabilization of the endoscope hard to achieve. Peristaltic movements in the duodenum can require medicating the patient to suppress such movements. Further, the tightly contracted musculature of the duodenal papilla may require precise maneuvering of a catheter through the papilla opening. Consequently, the effectiveness and efficiency of the procedure may become compromised, and the inability to cannulate the common bile duct may result in a failed or prolonged ERCP procedure and/or may result in procedural or post-procedural complications, such as infection.

Further, a colonoscope is a type of endoscope used for colonoscopies. Due to the length of the colonoscope and the tortuous anatomy of the colon, during some procedures, for example, during polyp removal, the colonoscope can become deformed or “loop” within the colon, causing pain for the patient and prolonging the procedure time. When looping occurs, the potential movements of the endoscopist may become limited. Consequently, it may become necessary to restart the colonoscopy procedure and the effectiveness and efficiency of the procedure may be compromised. Although endoscopists are trained in methods to reduce the prevalence of looping, it remains a recurring issue.

It is with the above considerations in mind that the improvements of the present disclosure may be useful.

### SUMMARY

The present disclosure, in its various aspects, is directed generally to expandable guide devices, implementation methods, and related systems. Embodiments according to the present disclosure, including those described herein, may increase particularly the effectiveness and efficiency of procedures used for the examination and treatment of conditions within the body, e.g., cannulation of the bile duct during an ERCP procedure or maneuvering within the colon during a colonoscopy procedure.

In an aspect, embodiments of the disclosure describe an expandable guide device that may comprise a first body comprising a proximal end, a distal end, the first body defining an instrument lumen extending therethrough along a longitudinal axis of the first body. The instrument lumen may be configured to slidingly receive a length of an instrument extendible therethrough. The expandable guide device may comprise a plurality of expandable members disposed circumferentially about the first body, each member of the plurality of expandable members of the first body extending between a proximal end and a distal end along the longitudinal axis of the first body. Each member of the plurality of expandable members of the first body may be independently expandable radially. Each expandable member may be independently expandable between an unexpanded configuration, partially expanded configuration, and expanded configuration, such that a first length of the instrument extendible through the instrument lumen of the first body is positionable radially, angularly, or both, relative to a central axis of a body lumen.

In various embodiments described herein and otherwise within the scope of the disclosure, wherein two or more of the plurality of expandable members of the first body are expandable, the expandable guide device may further comprise a plurality of first elongate fluid delivery members. Each first delivery member may be in fluid communication with a respective expandable member of the plurality of expandable members of the first body that are expandable. The expandable guide device may comprise a second body proximal to the first body, the second body may comprise a proximal end, a distal end, the second body defining an instrument lumen extending therethrough along a longitudinal axis of the second body. The instrument lumen may be configured to slidingly receive a second length of the instrument extendible therethrough. The second body may comprise a plurality of expandable members disposed circumferentially about the second body. Each member of the plurality of expandable members of the second body may be extending between a proximal end and a distal end along the longitudinal axis of the second body. Each member of the plurality of expandable members of the second body may be independently expandable radially between an unexpanded configuration, partially expanded configuration, and expanded configuration, such that the second length of the instrument extendible through the instrument lumen of the second body may be positionable radially, angularly, or both, relative to the central axis of the body lumen. Two or more members of the plurality of expandable members of the second body may be expandable and may further comprise one or more second elongate fluid delivery members. Each second delivery member may be in fluid communication with a respective expandable member of the plurality of expandable members of the second body that are expandable.

The plurality of expandable members may comprise a non-compliant material or a compliant material, or a combination thereof. The device may further comprise a handle and an actuating member may be attached at a proximal end to the handle and may be attached at a distal end to the first body, the second body, or both. The instrument lumen of the first body, the second body, or both, may be selectively slidably positionable along the respective first and second lengths of the instrument with the actuating member. The plurality of expandable members of the first body, the second body, or both, may comprise two, three, or four expandable members. The actuating member may comprise a lumen. The plurality of expandable members of the first body, the second body, or both, may be spherical, oblong, bean-shaped, kidney-shaped, finger-shaped or ellipsoidal, or any combinations thereof. Two or more members of the plurality of expandable members of the second body may be expandable and non-compliant. The one or more second elongate fluid delivery members may comprise a single elongate fluid delivery member in fluid communication with the two or more non-compliant expandable members.

In various embodiments described herein and otherwise within the scope of the disclosure, the expandable guide device may further comprise a second and third body aligned axially with each other and the first body, each comprising a proximal end and a distal end. The second body and the third body each may define an instrument lumen extending therethrough along a longitudinal axis of the second body and the third body. The instrument lumen of the second body and the third body may be configured to slidingly receive a respective second length and third length of the instrument extendible therethrough. The second body and the third body may comprise a plurality of expandable members disposed circumferentially about the first body, second body, and the third body. The expandable members may be extending from the proximal end of the second body to the distal end of the first body, and from the distal end of the second body to the proximal end of the third body, along the longitudinal axis of the first, second, and third body. The expandable members extending between the first and second bodies may be configured to expand radially from the longitudinal axis of the first and second bodies when an axial length between the first and second bodies is decreased. The expandable members extending between the first and second bodies may be configured to expand radially from the longitudinal axis of the second and third bodies when an axial length between the second and third bodies is decreased. The first, second, or third bodies, or combinations thereof, may be configured to be pushed or pulled along one or more of the longitudinal axes of the first, second and third bodies to selectively expand the expandable members between the first and second bodies, or the expandable members between the second and third bodies, or both. The plurality of expandable members of the first body may comprise a plurality of turns forming a coil, and wherein the coil may be disposed along the first body. The coil and the first body may be expandable radially when the distal end of the first body is fixed to the instrument and the proximal end of the first body is rotated about the longitudinal axis of the first body. The at least one expandable member may comprise a compliant material. The at least one expandable member may comprise one, two, three, or four expandable members. The at least one non-compliant expandable members may comprise one, two, three, or four expandable members.

In various embodiments described herein and otherwise within the scope of the disclosure, the actuating member may comprise a lumen. The at least one expandable member of the first body, the at least one non-compliant expandable member of the second body, or both, may be spherical, oblong, bean-shaped, kidney shaped, finger-shaped or ellipsoidal, or any combinations thereof. Two or more members of the plurality of expandable members of the second body may be expandable and non-compliant. One or more of the second elongate delivery member may comprise a single elongate fluid delivery member in fluid communication with the two or more non-compliant expandable members. The expandable guide device may further comprise a sleeve slidably disposed about the expandable guide device. The at least one expandable member of the first body, the second body, or both, may be adhered to the respective first body and the second body and may be arranged about the respective first body and second body with annularly symmetrical spacing. The second elongate fluid delivery member may comprise a single elongate fluid delivery member in fluid communication with a plurality of the non-compliant expandable members. The expandable guide device may be configured to be positioned proximal to an articulation joint of the instrument. The plurality of expandable members of the first body may comprise a shape memory metal or plastic material and may be formed from round wires, flat wire, or woven mech, or combinations thereof.

In an aspect, embodiments of this disclosure describe an endoscopic system that may comprise an instrument and an expandable guide device slidably disposed about the instrument. The expandable guide device may comprise a first body comprising a proximal end, a distal end, the first body may define an instrument lumen extending therethrough along a longitudinal axis of the first body, and the instrument lumen may be configured to slidingly receive a first length of the instrument extendible therethrough. The system may comprise a plurality of expandable members disposed circumferentially about the first body. Each member of the plurality of expandable members may be extending between a proximal end and a distal end along the longitudinal axis of the first body. Each member of the plurality of expandable members of the first body may be independently expandable radially between an unexpanded configuration, partially expanded configuration, and expanded configuration, such that the first length of the instrument extendible through the instrument lumen of the first body may be positionable radially, angularly, or both, relative to a central axis of a body lumen. The expandable guide device may comprise an actuating member and a handle may be attached to the first body. The handle may be configured to selectively position the instrument lumen of the first body along the length of the instrument by sliding the first body along the instrument with the actuating member.

In various embodiments described herein and otherwise within the scope of the disclosure, two or more members of the plurality of expandable members of the first body may be expandable, and further comprising a plurality of first elongate fluid delivery members. Each first delivery member may be in fluid communication with a respective expandable member of the plurality of expandable members of the first body that are expandable. The system may comprise a second body proximal to the first body. The second body may comprise a proximal end, a distal end, the second body may define an instrument lumen extending therethrough along a longitudinal axis of the second body, and the instrument lumen may be configured to slidingly receive a second length of the instrument extendible therethrough. It may comprise a plurality of expandable members disposed circumferentially about the second body. Each member of the plurality of expandable members of the second body may be extending between a proximal end and a distal end along the longitudinal axis of the second body. Each member of the plurality of expandable members of the second body may be independently expandable radially between an unexpanded configuration, partially expanded configuration, and expanded configuration, such that the second length of the instrument extendible through the instrument lumen of the second body may be positionable radially, angularly, or both, relative to the central axis of the body lumen. Each second elongate fluid delivery member may be configured to circulate fluid within the respective non-compliant expandable member. The first expandable guide device may be fixed by the first body, the second body, or both.

In various embodiments described herein and otherwise within the scope of the disclosure, the system may comprise one, two, three, four, five or more expandable members of the first body, the second body, or both, of the expandable guide device. Each expandable member may comprise a compliant or non-compliant material. The system may comprise an over-sleeve slidably disposed about the expandable guide device. The expandable member of the first body may comprise a compliant or a non-compliant material.

In an aspect, embodiments of the disclosure describe a method of performing an endoscopic procedure within a patient. The method may comprise inserting an endoscope into an instrument lumen of an expandable guide device, advancing the endoscope and the expandable guide device through a body lumen to a treatment site within the patient. The expandable guide device may include a plurality of expandable members disposed circumferentially about the expandable guide device, wherein each member of the plurality of expandable members may be independently expandable radially between an unexpanded configuration, partially expanded configuration, and expanded configuration. The method may comprise expanding one or more members of the plurality of expandable members at the first treatment site within the patient, such that the endoscope inserted through the instrument lumen of the expandable guide device is positionable radially, angularly, or both, relative to a central axis of the body lumen.

In various embodiments described herein and otherwise within the scope of the disclosure, the instrument may comprise an endoscope, such as a colonoscope, duodenoscope, bronchoscope, gastroscope, and the like. The plurality of expandable members may comprise a non-compliant material or a compliant material, or combinations thereof.

In various embodiments described herein and otherwise within the scope of the disclosure, the expandable guide device of the method may comprise one, two, three, four, five or more expandable members. The expandable member may be non-compliant. The expandable member may be compliant. Expanding the at least one expandable member may bend the endoscope. Expanding the at least one expandable member may straighten the endoscope. Expanding the at least one expandable member may stabilize the endoscope in position. The expandable members may be independently expanded. The method may comprise advancing a second device toward a treatment site. Expanding the at least one expandable member may adjust the position of the endoscope within the body lumen. Expanding the at least one expandable member may comprise inflation of the expandable member with air, CO₂, or fluid contrast.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1 illustrates an exemplary endoscope of a type described with respect to various embodiments of the present disclosure.

FIG. 2A illustrates a partial cross-sectional view of a system with an expandable guide device in an expanded configuration within a duodenum and an exemplary endoscope, such as the endoscope of FIG. 1, extended therethrough, in accordance with embodiments of the present disclosure.

FIG. 2B illustrates a partial cross-sectional view of a system with multiple expandable guide devices, such as the device of FIG. 2A, in a collapsed configuration within a body and an exemplary endoscope, such as the endoscope of FIG. 1, extended therethrough, in accordance with embodiments of the present disclosure.

FIG. 2C illustrates a partial cross-sectional view of a system with an expandable guide device in an expanded configuration within a duodenum and an exemplary endoscope, such as the endoscope of FIG. 1, extended therethrough, in accordance with embodiments of the present disclosure.

FIG. 3 illustrates a cross-sectional view of an expandable guide device, in an expanded configuration, according to embodiments of the present disclosure.

FIG. 4A illustrates a side view of an expandable guide device disposed along an instrument, such as the endoscope of FIG. 1, and FIG. 4B-4E illustrate cross-sectional views of the expandable guide device of FIG. 4A, in various configurations of expansion, according to embodiments of the present disclosure.

FIG. 5 illustrates a side view of a system with an instrument and an expandable guide device, according to embodiments of the present disclosure.

FIG. 6A illustrates a side-view of a system with an instrument and an expandable guide device, according to embodiments of the present disclosure. FIG. 6B illustrates a cross-sectional view of the expandable guide device of FIG. 6A.

FIGS. 7A-7B illustrate cross-sectional views of a system with an expandable guide device in an unexpanded and expanded configuration, respectively, according to embodiments of the present disclosure.

FIGS. 8A-8C illustrate cross-sectional views of a system with an expandable guide device in various configurations of expansion, according to embodiments of the present disclosure.

FIGS. 9A-9B illustrate perspective views of an expandable guide device in a respective un-pinched and pinched configuration, according to embodiments of the present disclosure.

FIGS. 10A-10B illustrate perspective views of a system with an expandable guide device bending an instrument within a body lumen in various configurations of expansion, according to embodiments of the present disclosure.

FIG. 11 illustrates a perspective view of a system with an instrument and an expandable guide device within a body lumen, in an expanded configuration, and a tool within an external tool channel, according to embodiments of the present disclosure.

FIGS. 12A-12B illustrate side views of an expandable guide device in various configurations of expansion, according to embodiments of the present disclosure.

FIGS. 13A-13B illustrate perspective views of an expandable guide device in various configurations of expansion, according to embodiments of the present disclosure.

FIGS. 14A-14B illustrate side views of an expandable guide device in various configurations of expansion, according to embodiments of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

It is the case, generally, that with any form of endoscopy or other procedure where any other instrument is inserted in the body it may be important to be able to stabilize the instrument in place once it is navigated to a desired position, to locate the instrument in a particular radial position defined by a cross-section of the body lumen, to orient an instrument in a particular manner dictated by a specific procedure, and the like.

Various embodiments according to the present disclosure are described below. As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

Embodiments of the present disclosure may include expandable guide devices for use with an instrument. An instrument may be an endoscope, bronchoscope, duodenoscope, colonoscope, gastroscope, ureteroscope, a catheter, or the like. The devices may include various components and configurations. Embodiments of this disclosure may comprise a medical device system. A medical device system may include an instrument, an expandable guide device, tools for use with the instrument, a proximal handle to, e.g., actuate the device, which extends to the body of the expandable guide device, and/or in embodiments including a balloon as an expandable member, a source of inflation fluid, or the like. Embodiments of the devices and systems may be used to substantially fill the space within a body lumen around the instrument, e.g., during an endoscopy, to enable the operator to stabilize the instrument within the body. Various embodiments described herein comprise an expandable guide device having plurality of expandable members about a body forming an instrument lumen that can slidingly receive a length of an instrument, such as an endoscope, within the instrument lumen. The expandable member(s) once expanded within the body lumen may advantageously inhibit or reduce movement of the instrument within the body lumen. In some embodiments, such as an expandable member comprising a balloon, the inflation fluid may be a gas, a liquid, or both. In some embodiments, the body lumen may include a lumen, organ, vessel, passage, or the like, within, e.g., the digestive system, or the like. In various embodiments, an expandable member may expand to a greater or lesser degree, may assume a different shape, and/or may exhibit different other properties than another expandable member. In various embodiments, the expandable guide device may be disposable. In alternate embodiments, the expandable guide device may be reusable.

ERCP procedures are used to examine, diagnose and treat areas in the common bile duct and pancreatic duct. An endoscope may be introduced into the patient, e.g., via the mouth, through the stomach and advanced through at least a portion of the small intestine. The endoscope may be used to access the ampulla of Vater in order to reach the pancreatic and bile ducts. The positioning, stabilization and/or angle of approach of the endoscope relative to the ampulla of Vater may be important to the success of the procedure and may be made difficult by movement and breathing of the patient, movement of the duodenum and operation of tools within or in relation to the endoscope. In order for a user to perform ERCP, the distal end of an instrument must be sufficiently stabilized within the body lumen at the ampulla or papilla of Vater, which requires extreme precision. Once placed in position, the instrument (e.g., endoscope) needs to be kept stable enough to facilitate access to and entry into and through the ampulla or papilla of Vater by the instrument and/or by an accessory device (e.g., biopsy tool) delivered through a working channel of the instrument and/or the expandable guide device.

Colonoscopies are a type of endoscopic procedure used to examine and treat the colon using an instrument (e.g., a colonoscope). The colonoscope, as an instrument with which an expandable guide device may be useful, is introduced into the patient via the anus and rectum, and through the colon. The instrument may be used to examine and diagnose tissue, take samples and/or remove polyps from the intestines during the examination. Due to the tortuous anatomy of the colon, when attempting to navigate the colonoscope through the curves of the colon, looping of the colonoscope, and consequently looping of intestines, can occur during the procedure, which may cause pain for the patient and may slow the procedure. Looping may cause the instrument to become undesirably positioned within the body lumen and require additional steps to be taken by the physician. The loop may be an alpha loop, a reverse alpha loop, an N loop, a reverse splenic flexure, a gamma loop, or a reverse sigmoid spiral. Current methods of resolving these loops involve proximal translation or removal of part or all of the instrument and restarting or continuing the procedure from the point at which the loop is no longer present, whereby the anatomy is substantially returned to a typical configuration. This process may cause additional discomfort and/or prolong the procedure further for the patient.

A number of medical procedures, including intravascular procedures, procedures along the digestive, urinary, respiratory, reproductive and/or biliary tracts, thoracic and pulmonary procedures, etc., utilize instruments, such as endoscopes, to access tissue intended for diagnosis or treatment (e.g., “target tissue”) within the body. In some instances, an endoscope may incorporate features which assist the physician in visualizing and performing a treatment on the tissue. For example, some endoscopes may include a light and/or camera designed to illuminate and/or visualize the body lumen as the endoscope is navigated and positioned adjacent to a target tissue site. Additionally, some endoscopes may also include a lumen (e.g., a working channel) through which a resecting device, grasping member, or other accessory devices may be deployed and utilized. Additional visualization and/or external and/or internal imaging methods may be alternatively or additionally employed, e.g., fluoroscopy.

Embodiments of the expandable guide devices, systems and methods of the present disclosure may be used with various endoscopes or other instruments, within different body lumens and with respect to a range of applications, such as described above, to enable the physician to perform procedures with greater precision, accuracy, and ease than without the expandable guide devices.

As used herein to describe the various embodiments described herein or otherwise within the scope of the disclosure, an inner surface of a body refers to a surface facing the instrument lumen and instrument when received therein. An outer surface of a body refers to a surface facing the body lumen.

In various embodiments described herein and otherwise within the scope of the disclosure, an expandable guide device may include a plurality of expandable members. The expandable members may be segmented or chambered and each segment or chamber may be independently expandable radially, e.g., to affect different shapes of the expandable member around the circumference of the device by maintaining unexpanded, or partially expanding, or expanding, various of the chambers of the expandable member. An expandable member may be a balloon, a mesh, a braid, a flap, a wire, or the like, and may be mechanically actuated, electrically actuated, pneumatically actuated, inflated, or the like. An expandable guide device may include one, two, three, four, five, or more expandable members. An expandable member may be adhered to a body, sheath, or the like, utilizing an adhesive, a tape, flowed material, welding (e.g., laser, ultrasonic, hot jaw thermal, etc.), melt/re-melt flow processes, or the like. An expandable member may be spherical, oblong, bean-shaped, kidney-shaped, finger-shaped or ellipsoidal, or any combinations thereof, or the like. An expandable member may be an expandable elastic material that may be compliant, non-compliant, or both, and may comprise latex, silicone, Pebax, a combination thereof, or the like. A compliant expandable member may be configured to expand to fill a body lumen. A non-compliant expandable member may be configured to expand to a set dimension and/or shape. An expandable member may be filled with fluid or gas. An expandable member may be transitioned from an unexpanded configuration to an expanded configuration (including a partially expanded configuration) to occlude, stretch, establish patency, or maintain patency of a body lumen. An expandable member may be used to center or position an instrument within the body lumen. An expandable member may be used to manipulate tissue of a body lumen for viewing or operation.

An expandable member or other portions of the device may comprise one or more radiopaque markers at various positions about and along the device. The radiopaque markers may be imaged during a procedure allowing, e.g., the position of the device or devices with respect to each other, with respect to the instrument, and/or with respect to a portion of the body lumen, to be determined, changed, confirmed, etc., and the like. The degree of expansion of the expandable members may be confirmed as well. Also, contrast fluid that is imageable may be injected in the body lumen or the inflation fluid for the expandable members may be made radiopaque. Radiopaque markers may include radiopaque filler compounded into the material of the expandable member and/or independent markers may be affixed to the interior or exterior of the expandable members, to the surfaces of the body, or to other portions of the device.

In various embodiments described herein and otherwise within the scope of the disclosure, an expandable guide device may include an actuating member. A proximal end of the actuating member may be manipulated to move the expandable guide device within a patient. The actuating member may have a lumen. The lumen may accept a stylet for manipulation and/or axial movement of the body with expandable members. The lumen for expansion and manipulation/movement may be separate or shared. Expandable members may be configured to be expanded in unison or may be configurate to be independently expanded. Expandable members may be expanded to different volume or degrees around the circumference of an expandable guide device and/or among expandable members on other expandable guide devices along the instrument, if desired, in order to affect orientation of the instrument, steer the instrument, and/or locate the instrument offset from the central axis, within the body lumen of the patient. The actuating members may be used to push and pull devices along the instrument. They may be used to position and/or reposition the device before expanding. The instrument lumen of the first body, second body, or both, may be selectively slidably positionable along the respective first and second lengths of the instrument with the actuating member.

Exemplary devices, systems, and methods with which embodiments of the present disclosure may be implemented include, but are not limited to, those described in the complete disclosures of U.S. Provisional Patent application having Attorney Docket No. 8150.0728Z, titled Devices, Systems, and Methods for Pyloric Occlusion, filed Aug. 27, 2020; U.S. Provisional Patent application having Attorney Docket No. 8150.0729Z, titled Stabilization and Leverage Devices, Systems, and Methods, filed Aug. 28, 2020; U.S. Provisional Patent application having Attorney Docket No. 8150.0722Z, titled Expandable Guide Devices, Systems, and Methods, filed even date herewith; and U.S. Provisional Patent application having Attorney Docket No. 8150.0723Z, titled Expandable Guide Devices, Systems, and Methods, filed even date herewith, each of which disclosures are herein incorporated by reference in their entirety.

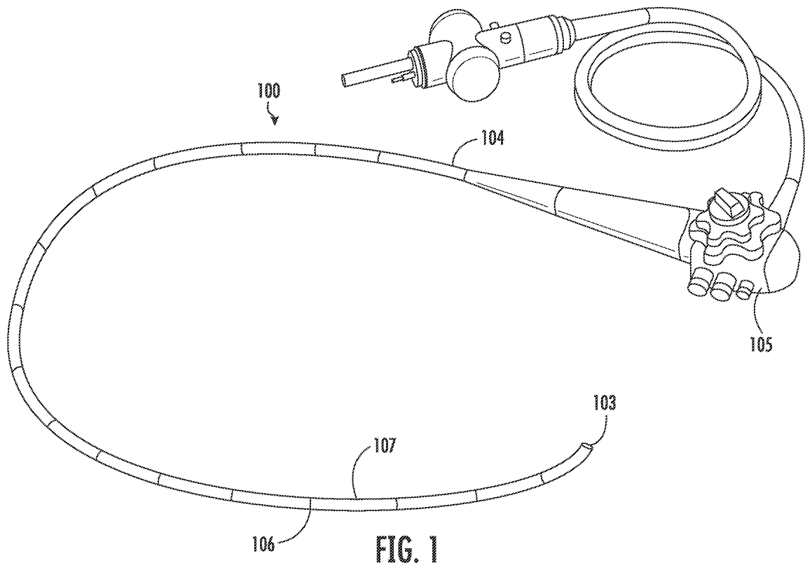

Examples of expandable guide devices for use with instruments are described herein with respect several embodiments. Referring to FIG. 1, an endoscope as an exemplary instrument of the type described in the present disclosure is illustrated. The instrument 100 is one example of an instrument that can be used with an expandable guide device described further herein. The instrument 100 (e.g., endoscope) comprises a distal end 103 and a proximal end 104 with a lumen or working channel extending therethrough. A handle 105 at the proximal end 104 may be operated by a medical professional to manipulate the instrument 100. The instrument 100 may include an articulation joint and/or cuts or channels 106 along a wall 107 of the insertion portion of the instrument 100 in order to facilitate movement and flexibility within a patient, e.g., including by operation of steering knobs at the handle 105.

In an exemplary method of performing an endoscopy, such as an ERCP procedure, a distal end of an endoscope may be advanced through the stomach and into the small intestine via the mouth of a patient. Once inside the patient, the digestive system may be visually examined and the pancreatic and bile ducts may be accessed using various tools passed through the working channel of the endoscope from the handle at the proximal end of the endoscope that remains outside of the body.

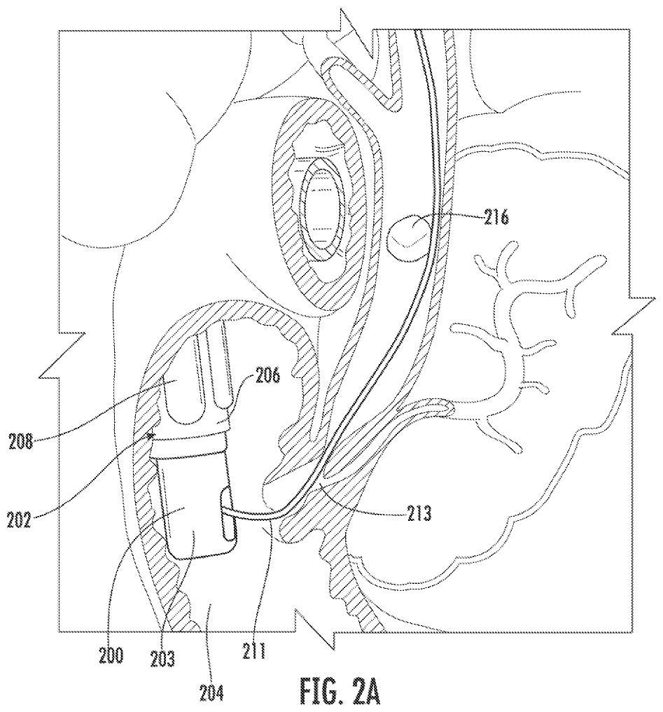

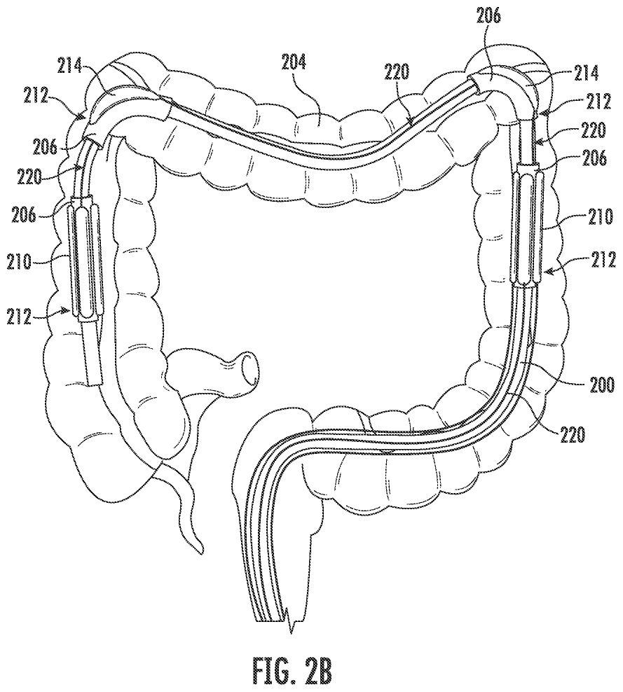

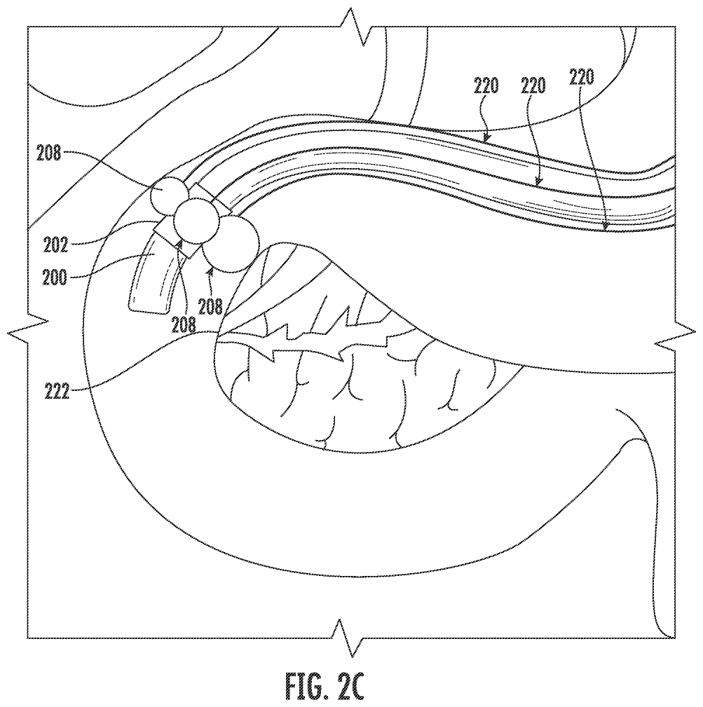

Examples of expandable guide devices for use with instruments, such as the endoscope of FIG. 1, are described. Referring to FIG. 2A, a partial cross-section view of a system according to embodiments of the present disclosure is illustrated. The instrument 200 (e.g., the instrument 100 of FIG. 1) is shown within a body lumen 204 (e.g., duodenum). A first expandable guide device 202 is shown at the distal end 203 of the instrument 200. A plurality of expandable members 208 (e.g., compliant or non-compliant balloons, or a mix) disposed on a body 206 of the expandable guide device are arranged about the circumference of the instrument in a partially expanded configuration. In FIG. 2A, the expandable guide device 202 is shown in the body lumen 204, with a tool 211 (e.g., catheter) extending from an exit of a working channel of the instrument 200. The tool 211 extends through an ampulla of Vater 213 to reach a blockage 216. In FIG. 2B, a plurality of expandable guide devices 212 are depicted, each with one of a plurality of expandable members 210, 214 partially expanded and disposed about a body 206, which defines an instrument lumen (not shown). For exemplary purposes, the body lumen 204 is the large intestine, including the colon. As may be seen, the colon presents twists and curves along its length that create challenging anatomy to navigate by simply steering the instrument 200 from outside the body. Each member of the plurality of expandable members 214 of the bodies 206 of the plurality of devices 212 is independently expandable radially between an unexpanded configuration, partially expanded configuration, and expanded configuration, such that lengths of the instrument 200 extendible through the instrument lumens (not shown) of the bodies is positionable radially, angularly, or both, relative to a central axis of the body lumen 204. The expandable members 210, 214 are expandable, and each expandable member is in fluid communication with a plurality of elongate fluid delivery members 220 independently or in series. The devices 212 may be positioned by the user prior to use outside a patient and/or moved along the instrument 200 once inside the patient. The devices 212 may be moved along the instrument by an actuating member (e.g., a stylet or the like, not shown), as described herein. As shown, the plurality of expandable guide devices 212 may assist in movement through portions of the body lumen 204 where positioning of the instrument 200 may be difficult. For example, the user may need to move through a portion of the body lumen 204 which forms a right angle. To move through this area, the expandable guide devices 212 may be used to force the instrument 200 to move in a particular direction. The device 212 may be moved into the right-angle portion of the body lumen 204 and a non-compliant expandable member 214 may be expanded. The non-compliant expandable member 214 may expand into the body lumen 204 and also expand into a fixed shape such that the body 206 is manipulated in the desired direction. Once the body 206 is in the desired position, the instrument 200 can be moved distally, through the body lumen 204. FIG. 2C shows another embodiment of a system according to the present disclosure. The instrument 200 is shown within a duodenum, near a duodenal papilla 222. In order to stabilize the instrument 200, the expandable members 208 of the expandable guide device 202 are expanded, pressing against the body lumen. In FIG. 2C, the expandable members 208 are differentially expanded by the elongate fluid delivery members 220. By selectively expanding and un-expanding individual expandable members 208, the user may radially move the position of the instrument 200 within the body lumen. In FIG. 2C, the expandable members 208 may be differentially expanded in order to stabilize the instrument 200 in the duodenum, orient the instrument 200 to a desired angle of approach of the working channel of the instrument 200 to the duodenal papilla 222, to better visualize the duodenal papilla 222, etc., or the like.

In various embodiments described herein and otherwise within the scope of the disclosure, an expandable member may be expanded in order to contact the body lumen, partially expanded when the user is positioning the device and does not want to unexpand the expandable member, and unexpanded when moving the device through the body lumen. The expandable members may be expanded independently so that the instrument may be moved radially within the body lumen. This radial movement may assist with stabilization, positioning, and movement of the device through difficult portions of the body lumen.

In various embodiments described herein and otherwise within the scope of the disclosure, an expandable guide device with pneumatically or expandable members may have a fluid inlet that may extend through the outer surface of an expandable member. The fluid inlet may supply and remove an inflation fluid for expanding and un-expanding or collapsing the expandable member. A single elongate fluid delivery member may be in fluid communication with a single expandable member, or with two or more expandable members, via the fluid inlets.

The one or more inflation fluid delivery members may extend along the instrument and/or along or through the body of an expandable guide device and through the outer surface of an expandable member. In some embodiments, an expandable member may comprise an expandable balloon, and the inflation fluid may be fluid, such as a gas, a liquid, or both. An inflation fluid delivery member may extend to the proximal end of the instrument and be connectable with a fluid source, e.g., a gas or liquid inlet/outlet at the handle of the guide device. Fluid (e.g., saline, air, etc.) may be inserted and removed from the inflation fluid delivery member by a source e.g., a syringe. The insertion of fluid may cause the expandable members to expand, and removal of fluid may cause the expandable members to un-expand, or collapse or deflate in the case, e.g., of a balloon as an expandable member. An inflation fluid delivery member may extend within a working channel or inflation lumen of an instrument. An inflation fluid delivery member may be in fluid communication with an inflation lumen of an instrument, while the instrument is extended within the instrument lumen of the body of an expandable guide device.

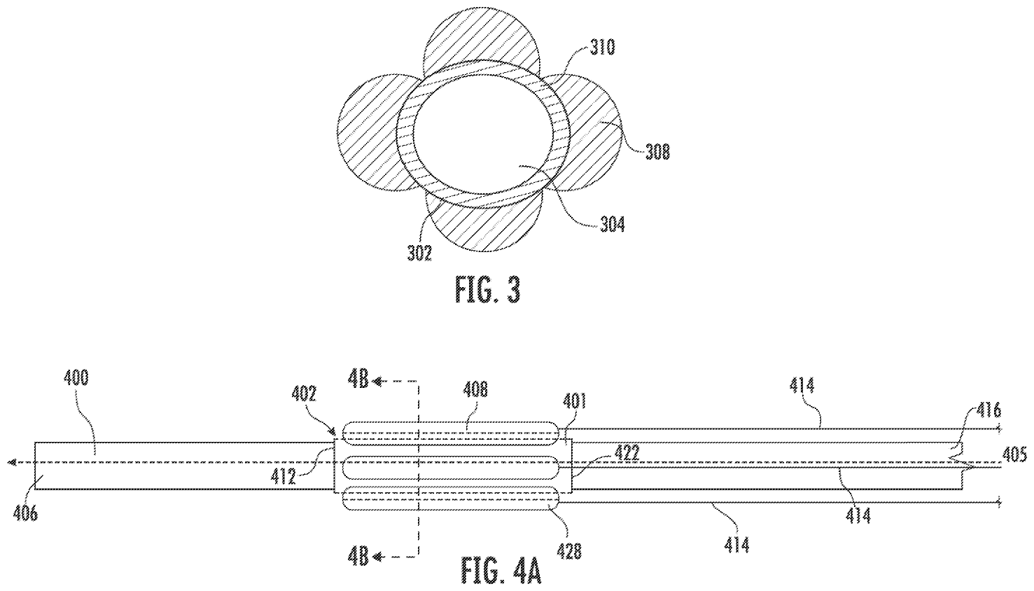

Referring to FIG. 3, a cross-sectional view of an expandable guide device is illustrated according to embodiments of the present disclosure. The device may be operated as described above. This view demonstrates placement of a plurality of expandable members 308 around a body 310, the body 310 defining an instrument lumen 304. The body 310 comprises an inward-facing surface forming the instrument lumen 304 and an outward-facing surface. The expandable members 308 may be disposed about the body 310 and expandable radially from the outward-facing surface. The device can be slidably disposed on an instrument received within the instrument lumen 304 of the body 310. The device can be fixed on the instrument. The device may be fixed manually prior to a procedure. The device may include an actuating member, with which the user may hold the device in place. The device may include a sheath that extends to the proximal end of the instrument, with which the user may hold the device in place. As shown, expandable members are partially expanded.

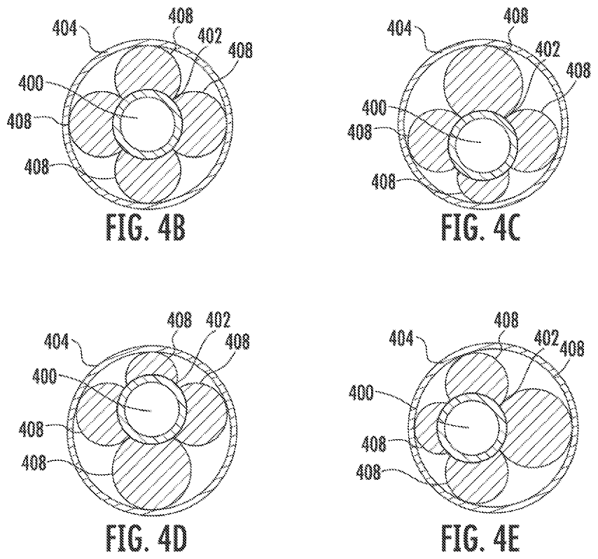

Referring to FIG. 4A, a side view of an expandable guide device 402 is illustrated according to embodiments of the present disclosure. The device 402 may be operated as described above. An expandable guide device 402 is depicted slidably disposed on an instrument 400 along a longitudinal axis 405 of the expandable guide device 402, near a distal end 406 of the instrument 400. The expandable guide device 402 includes a body 401 extending from a distal end 412 to a proximal end 422 of the body 401. The expandable members 408 extend axially from a distal end 412 to a proximal end 422 of the expandable guide device 402 and may be adhered to the body 401. Elongate fluid delivery members 414 extend from proximal ends 428 of the expandable members 408 toward a proximal end 416 of the instrument 400. An actuating member (not shown) may extend from the proximal end 422 of the body 401 toward the proximal end 416 of the instrument 400. When the expandable members 408 are in an expanded configuration, for example, such as the expandable members 308 in FIG. 3 (which are partially expanded), a fluid is delivered through the elongate fluid delivery members 414 to the expandable members 408. Each expandable member 408 may have its own dedicated delivery member 414, so that the expandable members 408 may be selectively expanded independent of the other expandable members 408 or selectively expanded along with combinations of other expandable members 408. Alternatively, two or more expandable members 408 may be expanded with the same delivery member 414, e.g., where expansion of the grouped expandable members 408 together is acceptable. In FIGS. 4B-4E, the embodiment of FIG. 4A is shown in a cross-sectional view with the expandable members 408 in various expanded arrangements. In FIG. 4B, the expandable members 408 are expanded equally within the body lumen 404. The instrument 400 and device 402 are centered and stabilized within the body lumen 404. In FIG. 4C-4E, the expandable members 408 are expanded independently in alternative configurations, allowing the instrument 400 and device 402 to be positioned radially at different positions offset from the central axis within the body lumen 404. In this way, the user has greater control over the instrument 400 and can position the instrument as necessary compared to navigating the instrument 400 without assistance of the expandable members 408. The expandable members 408 are depicted in an overall substantially spherical shape, but the expandable members 408 may be other shapes such as, e.g., cylinder, ellipse, a combination thereof, or the like. In some embodiments, the device is slidable along the instrument 400, such that the instrument 400 may be rotated or axially translated while the radial positioning is fixed. In certain embodiments, the device is fixed along the instrument 400.

In various embodiments described herein and otherwise within the scope of the disclosure, a stylet may also extend from a handle portion of the expandable guide device, e.g., kept at the proximal end of the instrument, through an actuating member or delivery member of the expandable guide device. The stylet can be used to push and/or pull the device in tandem with the instrument or relative to the instrument in order to position or reposition the device along the surface of the instrument. The stylet may hold the device in place when only the instrument is moved relative to the device. When moving the guide device with the stylet relative to the instrument or moving the guide device and the instrument in tandem, the expandable members can be partially expanded or unexpanded in the body lumen. The movement of the device along the instrument can be in a distal or proximal direction. Alternatively, the expandable member may be expanded, and the stylet is used to keep the device in place, facilitating movement of the instrument through the body lumen while the expanded expandable member keeps the body lumen stabilized. Two or more of a plurality of expandable members may be expanded in order to contact the body lumen, partially expanded when the user is positioning the device and does not want to unexpand the expandable member, and unexpanded when moving the device through the body lumen.

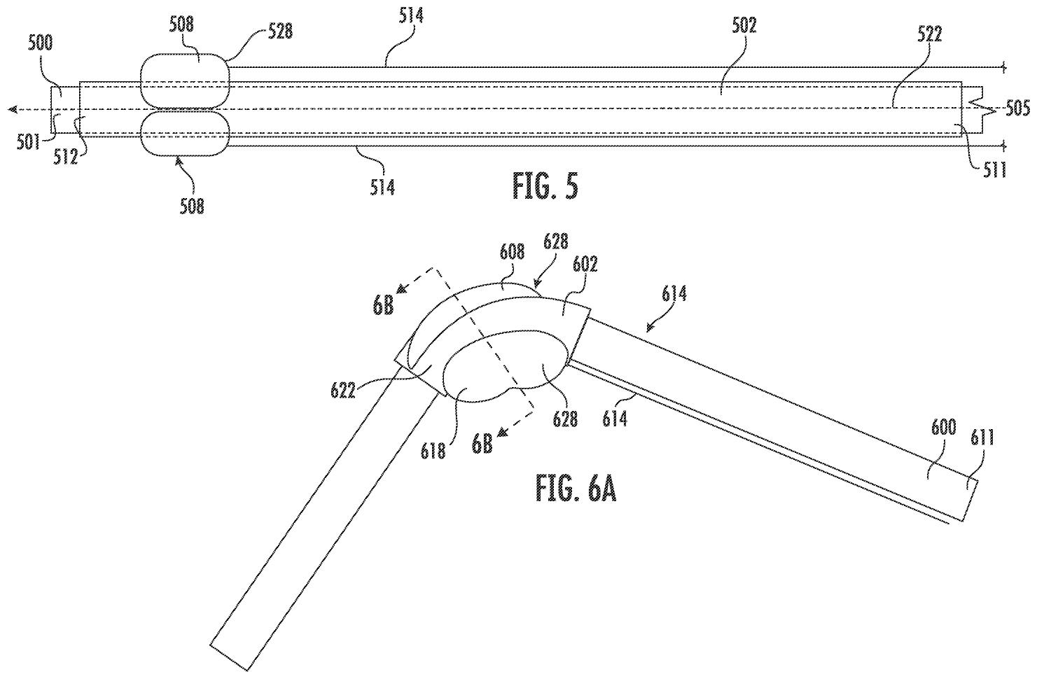

Referring to FIG. 5, a side view of an expandable guide device 502 is illustrated according to embodiments of the present disclosure. The device 502 may be operated similarly as described herein with various embodiments. The expandable guide device 502 with a body 522 is depicted slidably disposed about an instrument 500. The body 522 may be part of an elongate sheath having a distal end 512 that extends from a distal end 501 of the instrument 500 toward a proximal end 511 of the body 522 along the instrument 500. The instrument 500 is disposed along a longitudinal axis 505 of the expandable guide device 502 within an instrument lumen of the body 522 (not shown). The instrument lumen and a longitudinal axis 505 may be coextensive with each other or may be radially offset from each other. An inner surface (not shown) of the body 522 maintains frictional contact with the instrument 500. The body 522, as well as embodiments of bodies of other expandable guide devices described here or otherwise, may be formed from a tube of a material, e.g., silicone, latex, low density polyethylene (LDPE), high density polyethylene (HDPE), nylon, nylon 6, nylon 6,6, thermoplastic polyurethane (TPU), polyether block amide, polytetrafluoroethylene (PTFE) or fluorinated ethylene propylene (FEP) Teflon, or the like, which does not substantially change in dimension. The instrument lumen is configured to receive the instrument 500, e.g., an endoscope, slidingly extendable through the instrument lumen. A plurality of expandable members 508 extend along the longitudinal axis 505 and are disposed radially around the distal end 512 of the body 522. Elongate fluid delivery members 514 extend through proximal ends 528 of the plurality of expandable members 508 and along the body 522 (e.g., sheath) of the device 502 toward a proximal end of the instrument 500. Separate elongate fluid delivery members 514 facilitate the expansion of the expandable members 508 to be independent, with controlled, variable expansion. Alternatively, two or more expandable members 508 may share a common fluid delivery member 514, if their expansion is coincident with the other sharing delivery members 508. Inflation fluid may be supplied through the elongate fluid delivery members 514 to transition the expandable members 508 between an unexpanded configuration and an expanded configuration (including a partially expanded configuration). FIG. 5 depicts the plurality of expandable members 508 as partially expanded. The body 522 may carry multiple fluid delivery members 514 within or along the body 522, and may be used to organize the delivery members 514 radially about the instrument 500 to prevent cross-over or tangling. Further, the body 522 may be translated axially along the instrument 500 to position or reposition the expandable guide device 502 relative to the instrument 500 and/or the body lumen. In some embodiments, the device 502 is slidable along the instrument 500, such that the instrument 500 may be rotatable and/or axially translatable with respect to the expandable members 508 while radial positioning of the instrument 500 in the body lumen is dictated by the expansion, partial expansion or non-expansion of the expandable members 508 about the instrument 500, as described above. In some embodiments, the device 502 may be immovable at a selected position relative to the instrument 500.

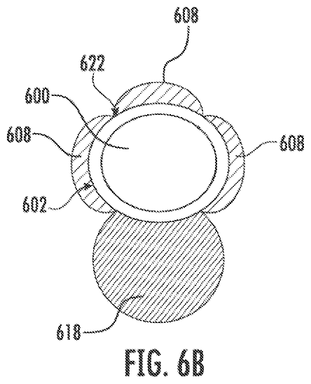

Referring to FIG. 6A, a side view of an expandable guide device 602 is illustrated according to embodiments of the present disclosure. The device 602 may be similarly operated as described with various embodiments herein. The expandable guide device 602 has a body 622 depicted slidably disposed about an instrument 600. Compliant 608 and non-compliant 618 expandable members are disposed about (e.g., adhered to) the body 622 of the expandable guide device 602. FIG. 6A depicts the compliant expandable member 608 as unexpanded, and the non-compliant expandable member 618 as expanded. Elongate fluid delivery members 614 extend to proximal ends 628 of the plurality of expandable members 608, 618 and extend toward a proximal end 611 of the instrument 600. In this configuration, the noncompliant expandable member 618 is expandable to a predetermined shape (e.g., spherical, oblong, bean-shaped, kidney-shaped, finger-shaped, ellipsoidal, and the like). This predetermined shape may be used to orient the instrument 600 as the user desires, as shown in FIG. 6A to be a substantially perpendicular bend. This movement of the instrument 600 may assist the user in navigating through curved and angled portions of a body lumen, e.g., such as is shown in FIG. 2B. The expandable members 608, 618 assist in preventing loops from occurring or resolving loops that may have occurred, as described above, for example, with respect to FIGS. 4A-4B. FIG. 6B illustrates a cross-sectional view of the bent instrument 600 and expandable guide device 602 of FIG. 6A from along the longitudinal axis of the body. As in FIG. 6A, the noncompliant expandable member 618 is expanded and the compliant expandable members 608 are not expanded. In other embodiments, the expandable guide device 602 may have two, three, four, or more non-compliant expandable members 618 and/or compliant expandable members 608, which may be selectively expandable to manipulate the direction of the instrument 600. In some embodiments, the device 602 may be slidable along the instrument 600, such that the instrument 600 may be rotated or axially translated with respect to the expandable members while radial positioning of the instrument 600 in the body lumen is dictated by the expansion, partial expansion or non-expansion of the expandable members 608, 618 about the instrument 600, as described above. In other embodiments, the expandable guide device 602 may be rotated or axially translated relative to the instrument 600, while the radial positioning is fixed. Movement may be achieved by manipulating a sheath or elongate member (e.g., stylet) (not shown) attached to the body 622 and running the length of the instrument to the proximal end 611. In some embodiments, one or more fluid delivery members (e.g., 614) may be purposed to also facilitate movement of the guide device 602 with respect to the instrument 600. In other embodiments, the device 602 is fixed along the instrument 600.

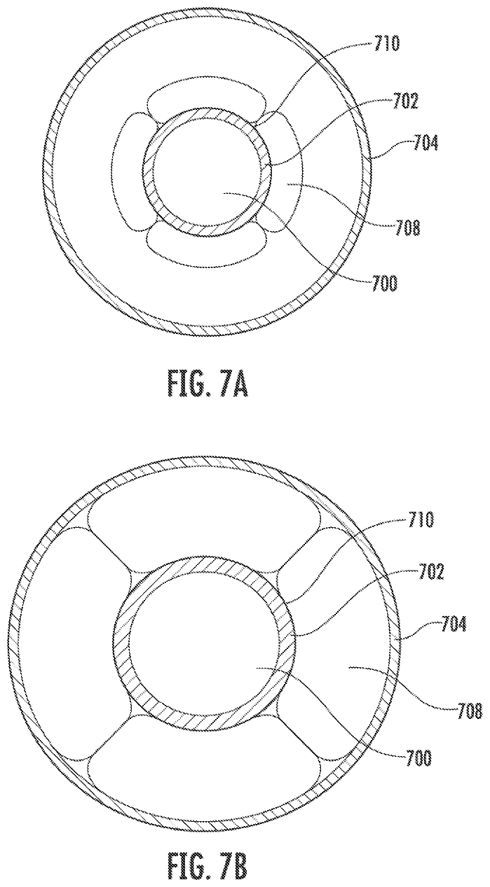

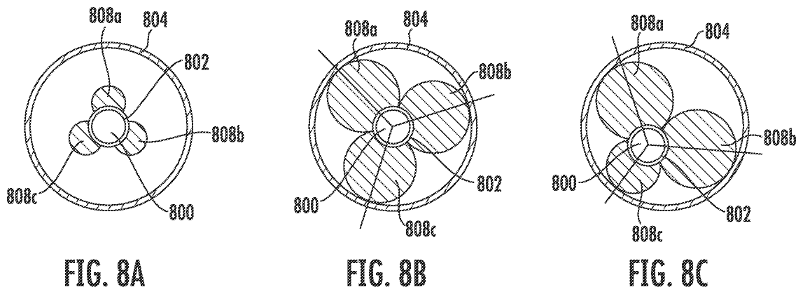

Referring to FIGS. 7A and 7B, cross-sectional views of an expandable guide device are illustrated according to embodiments of the present disclosure. The device may be operated as described above. A body 702 is depicted slidably disposed about an instrument 700. FIG. 7A illustrates expandable members 708 disposed about the body 702 in an unexpanded configuration and FIG. 7B illustrates the expandable members 708 in a symmetrically expanded configuration. In the expanded configuration of FIG. 7B, expandable members 708 are expanded within a body lumen 704. The plurality of expandable members 708 may be adhered to the outer surface 710 of the body 702 such that the plurality of expandable members 708 do not overlap. In the position of FIG. 7B, the expanded expandable members 708 center the instrument 700 within the body lumen 704, stabilizing the instrument 700. This embodiment allows the user to use the instrument 700 without the instrument 700 shifting, which may be exacerbated by movement of the body lumen 704. In another embodiment, the expandable members 708 may be expanded to different levels of expansion, such that the instrument 700 may be off-centered with respect to a central axis of the body lumen 704 (see e.g., FIGS. 8A-8C). Off-centering an instrument 700 by expandable members 708, may facilitate establishing a desired positioning and/or angle of approach of a distal end of an endoscope to a target tissue, organ, or adjacent body lumen 704 for performing a procedure. In this embodiment, the body 702 is fixed about the instrument 700. In some embodiments, the device is slidable along the instrument 700, such that the instrument 700 may be rotated or axially translated while the radial positioning is fixed. In embodiments where movement of the body of the guide device relative to the instrument 700 is desired, such movement may be achieved, for example, as described above with respect to FIGS. 6A-6B.

Referring to FIGS. 8A-8C, cross-sectional views of an expandable guide device 802 are illustrated according to embodiments of the present disclosure. The device 802 may be similarly operated as described in various embodiments herein. An expandable guide device 802 is depicted slidably disposed about an instrument 800. FIG. 8A illustrates the expandable guide device 802 in an unexpanded configuration. FIG. 8B illustrates the expandable guide device 802 with expandable members 808a, 808b, 808c expanded within a body lumen 804. FIG. 8C illustrates the expandable guide device 802 in a partially expanded configuration, with expandable members 808a, 808b expanded and expandable member 808c partially expanded. In the depiction of FIG. 8C, the expandable members 808a, 808b position the expandable guide device 802 and instrument 800 in a selected direction. This movement can be used to position the expandable guide device 802 and instrument 800 relative to the body lumen 804 by the user. This may allow a user to position a distal end of an endoscope as desired relative to a target tissue for performing a procedure. For example, in this embodiment and in other embodiments, an opening of a working channel may be positionable by a user such that additional tools extending through the working channel of the instrument 800 may have sufficient or optimal angle of approach and/or working space for performing the procedure. The instrument 800 may be positioned radially and fixed by controlled expansion of the expandable members 808a, 808b, 808c by the user, within the body lumen 804, allowing for greater stability and control over the placement of the instrument 800. In some embodiments, the device 802 is slidable along the instrument 800, such that the instrument 800 may be rotated or axially translated while the radial positioning is fixed. In embodiments, the device 802 is fixed along the instrument 800. In embodiments where movement of the body of the guide device 802 relative to the instrument 800 is desired, such movement may be achieved, for example, as described above with respect to FIGS. 6A-6B.

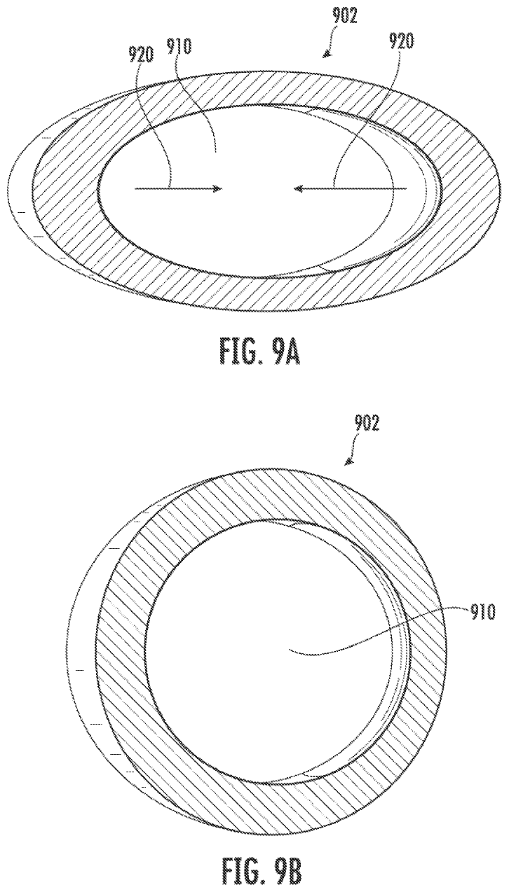

Referring to FIGS. 9A-9B, perspective views of an expandable guide device 902 are illustrated according to embodiments of the present disclosure. A body of the expandable guide device 902 is depicted in a relaxed configuration, FIG. 9A, as an ellipsoidal ring with an instrument lumen 910 therethrough. The expandable guide device 902 may be compressed (e.g., un-relaxed), as indicated by arrows 920, resulting in the compressed configuration of FIG. 9B, which may be moved slidably about and along an instrument (not shown). This embodiment illustrates how the expandable guide device 902 may be placed on an instrument and stay fixed to the instrument once it is placed in the desired location along the instrument and allowed to assume the resting position of FIG. 9A. Expansion members (not shown) may be disposed about the body of the device 902, and configured in shape, dimensions, materials, etc., and operated, as described above in the previous embodiments.

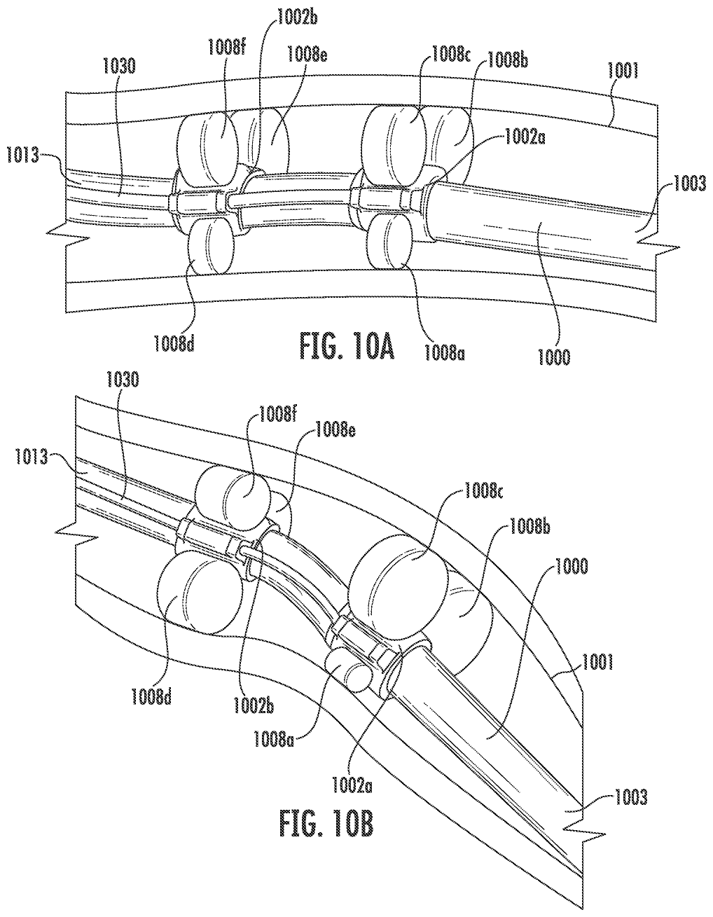

Referring to FIGS. 10A-10B, perspective views of a system including an instrument 1000 and a plurality of expandable guide device 1002a, 1002b are illustrated according to embodiments of the present disclosure. The devices 1002a, 1002b may be similarly operated as described with various embodiments herein. In these figures, first and second expandable guide devices 1002a, 1002b are depicted slidably disposed on an instrument 1000. A first device 1002a is positioned toward a distal end 1003 of the instrument 1000, and a second device 1002b is positioned toward a proximal end 1013 of the instrument 1000. In FIG. 10A, devices 1002a and 1002b each have two respective expandable members 1008b, 1008c, 1008e, 1008f in an expanded configuration and one expandable member each, 1008a, 1008d, in a partially expanded configuration, within a body lumen 1001. In this configuration, the instrument 1000 can be positioned radially within the body lumen 1001, as seen in FIG. 10B. FIG. 10B depicts the instrument 1000 moving through a curved portion of the body lumen 1001. To assist the instrument 1000, the expandable members are inflated or expanded such that the first device 1002a has two expandable members 1008b, 1008c expanded against the body lumen 1001, with one expandable member 1008a much less expanded compared to the other expandable members 1008b, 1008c of device 1002a, and the second device 1002b has one expandable member 1008d expanded against the body lumen 1001, with two expandable members 1008e, 1008f less expanded compared to the other expandable member 1008d of the device 1002b. In this configuration, the expandable guide devices 1002a, 1002b may be controlled to move the instrument 1000 into an angled position, easing movement through the body lumen 1001. In this embodiments and others. The expandable members 1008a, 1008b, 1008c, 1008d, 1008e, 1008f may also aid in stabilizing an endoscope in a body lumen 1001 by providing a substantial barrier circumferentially around the endoscope, so that the expandable members 1008a, 1008b, 1008c, 1008d, 1008e, 1008f suspend the endoscope. For example, having a plurality of expandable members 1008a, 1008b, 1008c, 1008d, 1008e, 1008f positionable between a body lumen 1001 wall and the endoscope, peristaltic motion or other body movements may be buffered by the expandable members 1008a, 1008b, 1008c, 1008d, 1008e, 1008f, thereby allowing the endoscope (including imaging and illumination components) to remain in a desired position for performing a procedure. In the embodiment of FIGS. 10A-10B, the expandable guide device may also comprise elongate fluid delivery members in fluid communication with one or more of the plurality of expandable members. The elongate fluid delivery members may extend through an external working channel 1030 of the second device 1002b extending along the surface of the instrument 1000 in order to provide fluid to the plurality of expandable members 1008a, 1008b, 1008c of the first device 1002a. In some embodiments, the two devices 1002a, 1002b may be connected by a working channel, which also may hold elongate fluid delivery members. In some embodiments, each device is slidable along the instrument, such that the instrument may be rotated or axially translated while the radial positioning is fixed. In certain embodiments, each device is fixed along the instrument. In some embodiments, one device may have a body with expandable members at the distal and proximal ends of the device, with the distal and proximal ends connected by an external working channel. In embodiments where movement of the body of the guide devices relative to each other and/or the instrument is desired, such movement may be achieved, for example, as described above, e.g., with respect to the devices of FIGS. 6A-6B.

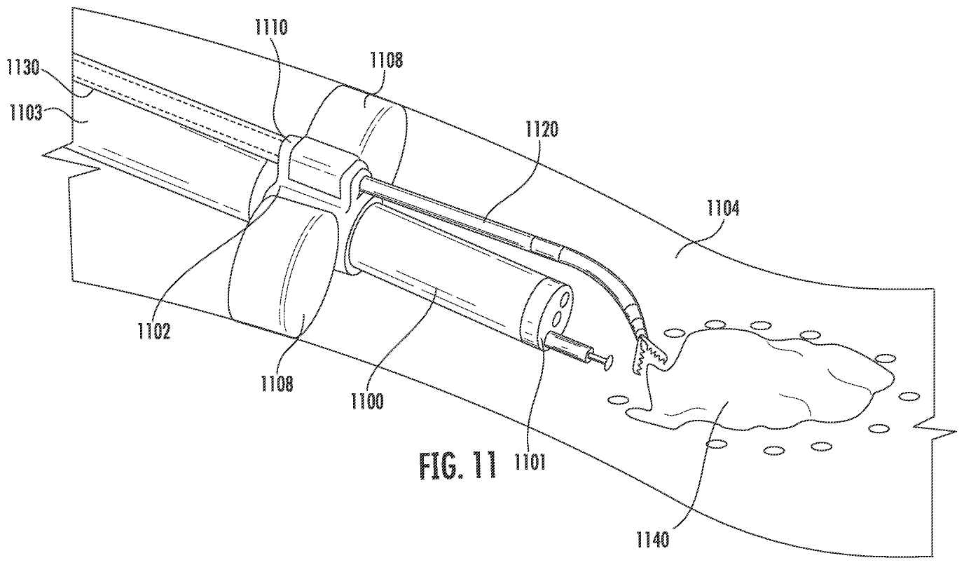

Referring to FIG. 11, a perspective view of a system including an instrument 1120 and expandable guide device 1102 is illustrated according to embodiments of the present disclosure. The device 1102 may be operated as described above. In this embodiment, an expandable guide device 1102 is depicted slidably disposed on the instrument 1100. The expandable guide device 1102 is near a distal end 1101 of the instrument 1100. A plurality of expandable members 1108 are expanded within a body lumen 1104. This configuration helps to stabilize the instrument 1100 within the body lumen 1104. The expandable guide device 1102 further comprises a working channel 1130, external to the instrument 1100 extending through a channel guide 1110 of the device 1102 and toward a proximal end 1103 of the instrument 1100. The working channel 1130 may be used to hold a tool 1120, such as a grasping device for manipulating tissue, elongate fluid delivery members to deliver fluid to the plurality of expandable members 1108, a stylet to direct movement of the expandable guide device 1102, or the like. The tool 1120 may be rotatable and axially translatable in order to achieve the desired position and angle of approach to a target area. The tool may also be rotatable and axially translatable with respect to a second tool (not shown) which may be passed through a channel of the instrument (not shown). The tool 1120 may be steerable at its tip. The working channel 1130 may extend to the handle of the device at the proximal end of instrument 1100 (seen in FIG. 1). The expandable members 1108 may be selectively expanded or unexpanded to change the angle of the device 1102 and instrument 1100 with respect to the target area. In this embodiment, the user may use the expandable guide device 1102 to stabilize the instrument 1100 and, upon finding a lesion 1140, use the expandable guide device 1102 to position the instrument such that the tool 1120 can grasp a sample of the lesion 1140 for analysis. As shown, in one example, the grasping tool may be delivered through the external working channel of the expandable guide device extending along the outside of the instrument, while a RF conductive needle knife may be extended along the internal working channel of the instrument and manipulated to resect tissue from the lesion. Other tools deliverable via the working channel 1130 of the guide device and/or through the working channel of the instrument 1100, are contemplated. For example, a tissue retraction device may be incorporated as part of the system, such as an elastic tether with fasteners, e.g., clips, at either or both ends of the tether. The tether may be fastened to the lesion 1140 or surrounding tissue at one end of the tether and to the wall of the body lumen at the other end of the tether. The elastic tether may be stretched to apply tension to the tissue, while it is being dissected/resected. In some embodiments, the device is slidable along the instrument, such that the instrument may be rotated or axially translated while the radial positioning is fixed. In various embodiments, the device 1110 may be fixed along the instrument 1100.

In combination, with this embodiment and other embodiments described, a system having an instrument and one or more expandable guide devices with expandable members provides the capability of a stable platform for the instrument once in position for diagnostic or treatment procedures to be performed with multiple, simultaneous tool access to the treatment site.

In various embodiments described herein and otherwise within the scope of the disclosure, a stylet may also extend from a handle portion of the guide device, e.g., kept at the proximal end of the instrument, through an actuating member or delivery member of the device. The stylet can be used to push and/or pull the device or portions of the device relative to the instrument in order expand or unexpand the device along the surface of the instrument. The stylet may hold the device in a chosen configuration. When moving the device with the stylet relative to the instrument or moving the guide device and the instrument in tandem, the expandable member may not be expanded. The movement of the device along the instrument can be in a distal or proximal direction. Alternatively, when the expandable member is expanded and the stylet may be used to keep the device in place, facilitating movement of the instrument through the body lumen while the expanded expandable member keeps the body lumen stabilized. An expandable member may be expanded in order to contact the body lumen, partially expanded when the user is positioning the device and does not want to unexpand the expandable member, and unexpanded when moving the device through the body lumen.

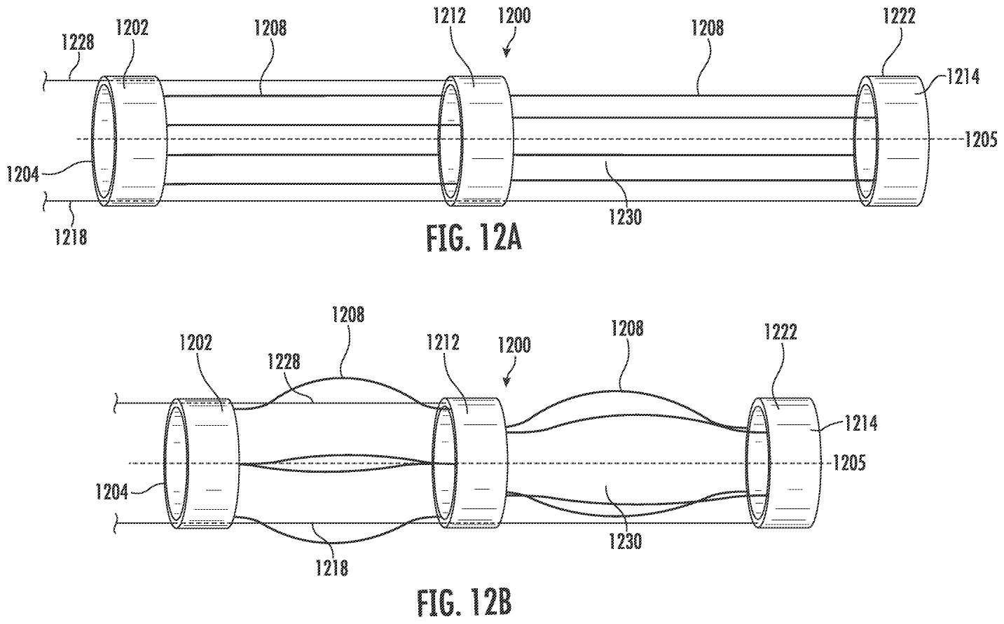

Referring to FIGS. 12A-12B, side views of an expandable guide device are illustrated according to embodiments of the present disclosure. In FIG. 12A, an expandable guide device 1200 is depicted with three bodies 1202, 1212, 1222 connected by a plurality of expandable members 1208, with the bodies aligned axially. The plurality of expandable members 1208 may be round, square, rectangular or flat wires, strips of metal or polymers, or the like. The expandable guide device 1200 also has actuating members 1218, 1228. The first actuating member 1218 is coupled to the third body 1222 and is slidably disposed within the second body 1212 and the first body 1202. The second actuating member 1228 is fixed to the second body 1212 and is slidably disposed within the first body 1202. An instrument may be slidably disposed within an instrument lumen 1230 such that the instrument lumen is configured to slidingly receive a first, second, and third length of the instrument therethrough. As illustrated by FIG. 12B, when the first actuating member 1218 is pulled proximally, the third body 1222 moves toward a proximal end 1204, reducing the distance along a longitudinal axis 1205 between the third body 1222 and the second body 1212. The plurality of expandable members 1208 between the third body 1222 and the second body 1212 expand radially away from the longitudinal axis 1205. When the second actuating member 1228 is pulled proximally away from a distal end 1214, the second body 1212 moves toward the proximal end 1204, reducing the distance along the longitudinal axis 1205 between the second body 1212 and the first body 1202. The plurality of expandable members 1208 between the second body 1212 and the first body 1202 expand radially away from the longitudinal axis 1205. The first body 1202 is fixed in position on the instrument as a stop against which the actuating members may pull in order to expand the expandable members between first and second bodies, or second and third bodies, or both. Alternatively, in another embodiment, the third body 1222 is fixed on the instrument, the first actuating member 1218 is adhered to the second body 1212 and slidably disposed within the first body, and the second actuating member 1228 is adhered to the first body 1202. In this embodiment, when the first actuating member 1218 is pushed, the second body 1212 moves distally toward the third body 1222, and when the second actuating member 1228 is pushed, the first body 1202 moves distally toward the second body 1212. In another embodiment, the shape memory resting state of the plurality of expandable members 1208 may be in an expanded configuration. In this embodiment, a sheath or over-sleeve would be disposed over the expandable guide device 1200 until arriving at the desired location within the body, and then the sheath could be retracted, exposing the plurality of expandable members 1208, allowing the expandable members to self-expand to the resting state. Alternatively, in this embodiment, the actuating members 1218, 1228 could be holding the first body 1202 and second body 1212 taught, and once in position, the actuating members 1218, 1228 would be loosened, allowing the plurality of expandable members 1208 to self-expand. In these embodiments, the plurality of expandable members 1208 may be expanded into a body lumen, stabilizing and positioning (e.g., centering) the instrument within the body lumen at a chosen position. The first, second, or third bodies, or combinations thereof, are configured to be pushed or pulled along one or more of the longitudinal axes of the first, second and third bodies to selectively expand the expandable members between the first and second bodies, or the expandable members between the second and third bodies, or both.

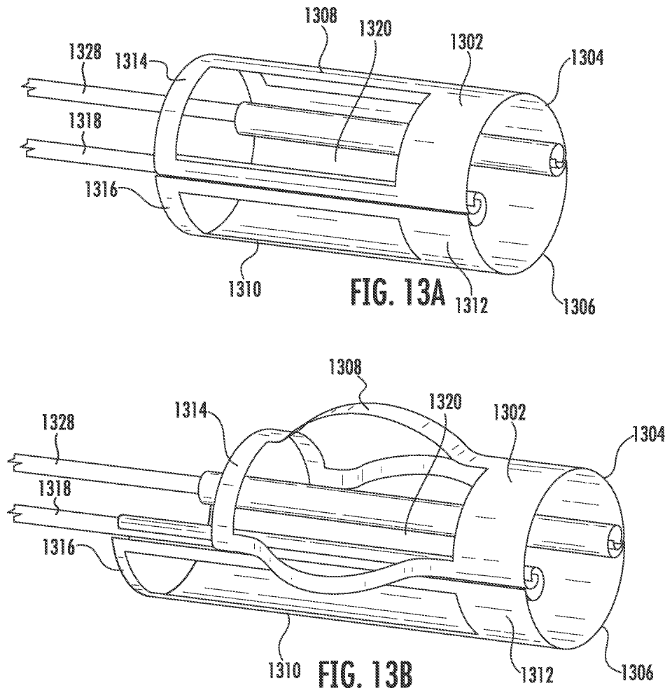

Referring to FIGS. 13A-13B, perspective views of an expandable guide device are illustrated according to embodiments of the present disclosure. In the embodiment of FIG. 13A, a first body 1302 is depicted with a first distal end 1304 and a first proximal end 1314, with a plurality of expandable members 1308 extending between the first distal end 1304 and first proximal 1314 ends. A second body 1312 has a second distal end 1306 and a second proximal end 1316, with a plurality of expandable members 1310 extending between the second distal end 1306 and second proximal end 1316. The first distal end 1304 and second distal end 1306 and the first proximal end 1314 and second proximal end 1316 are interlocked such that holding the second distal end 1306 and second proximal end 1316 in a fixed position and moving the first proximal end 1314 toward the first distal end 1304, the first proximal end 1314 slides along the portion interlocked with the second proximal end 1316. In this movement, the plurality of expandable members 1308 expand radially, as seen in FIG. 13B. This movement can be made with a first rod 1318, which is coupled to the first proximal end 1314. A second rod 1328 is coupled to the second proximal end 1316. The distal ends 1304, 1306 can be fixed. Either the first rod 1318 or second rod 1328 can be used to move the respective proximal end toward the respective distal end 1304, 1306 expanding the expandable members 1308, 1310. When using one rod 1318, 1328 the other may remain fixed. If both distal ends are fixed, both rods 1318, 1328 may be used to expand both bodies 1302, 1312 simultaneously by pulling proximally against a fixed point of the proximal ends 1314, 1316 of the bodies 1302, 1312. An instrument can be slidably disposed within the instrument lumen 1320. The expansion of the expandable members 1308, 1310 radially positions the instrument within a body lumen, as the expandable members 1308, 1310 expand into the body lumen.

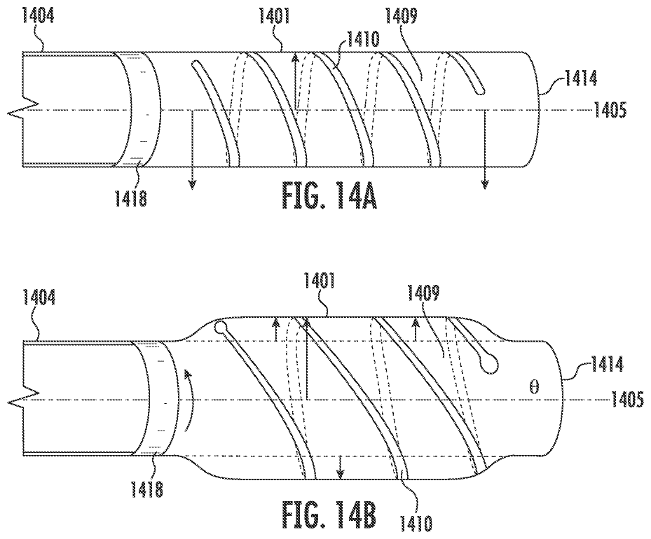

Referring to FIGS. 14A-14B, side views of an expandable guide device are illustrated according to embodiments of the present disclosure. In the embodiment of FIG. 14A, a body 1401 is depicted, extending from a distal end 1414 to a collar 1418 proximal to the distal end 1414. The collar 1418 extends proximally to a proximal end 1404 of the device and an instrument (not shown). Between the collar 1418 and the distal end 1414, a coiled body 1410 is enmeshed with the body 1401. The body 1401 may comprise a compliant material, such as Latex, or silicone, that is stretchable with the coiled body 1410 as it is expanded. The coiled body 1410 may be a shape memory material, such as Nitinol, and the like. In FIG. 14A, the coiled body 1410 is tightly wound. In FIG. 14B, the coiled body 1410 is relaxed. The distal end 1414 of the body 1401 is fixed to the instrument (e.g., by interference friction fit, clamp, and the like) in order to provide a stop against which the coiled body 1410 may be rotated to expand the coiled body 1410 and the body 1401 of the device. In an embodiment, the coiled body 1410 moves from tightly wound to relaxed by rotation of the collar 1418 around the longitudinal axis 1405. In another embodiment, the coiled body 1410 moves from tightly wound to relaxed (e.g., expanded) by removal of a sheath. An instrument can be slidably disposed within the instrument lumen 1409. When the coiled body 1410 relaxes, this expands the body 1401. In a further embodiment, the coiled body 1410 moves from a relaxed to an expanded state by rotation of the collar 1418, so that when the user disengages the collar 1418 the coiled body 1410 wants to revert to the unwound, relaxed state. Expansion of the body 1401 may be used to stabilize an instrument within the body lumen.

In various embodiments described herein and otherwise within the scope of the disclosure, the expandable members have sensors for measuring temperature, pressure, and the like. The sensors may alert the user as to when the expandable member has expanded into the body lumen wall. The sensors may further inform the user when to stop expanding the expandable member based on a reading of pressure or temperature. The sensor may trigger an automatic response, stopping expansion once a threshold is reached.

In various embodiments described herein and otherwise within the scope of the disclosure, an expandable member may have an expanded configuration (including partially expanded configuration) and a collapsed configuration. An expandable member may comprise a variety of compliant, semi-compliant, or non-compliant materials. These materials may comprise silicone, latex, polyurethane, rubber, isobutylene or the like. The thickness of the expandable member may vary with the material and may relate to the outer diameter of the expandable member in the collapsed and the expanded configuration. An expandable member may be expanded and collapsed or otherwise stretched once or a plurality of times to increase its elasticity prior to use within a patient, which may improve a symmetrical inflation of the expandable member and may improve the centering mechanics of the expandable member. An expandable member may be expanded via a supply of an inflation fluid through the fluid delivery members and one or more fluid inlets that are part of the expandable members. The same fluid delivery member and inlet may be used to expand and deflate the expandable member. Alternatively, a supply fluid inlet and a return fluid inlet may be intermittently or continuously used to circulate inflation fluid through an expandable member. A continuous flow of inflation fluid through a supply fluid inlet and a return fluid inlet may substantially maintain a desired pressure of the inflation fluid within the expandable member, or the inflation fluid may be heated and circulated to maintain a desired temperature within the expandable member.

In various embodiments described herein and otherwise within the scope of the disclosure, the distal end of the instrument may or may not extend past the distal end of the expandable guide device.

In various embodiments described herein and otherwise within the scope of the disclosure, an expandable member may expand to a greater or lesser degree than other expandable members.

In various embodiments described herein and otherwise within the scope of the disclosure, the expandable guide device may be disposable. In alternate embodiments, the expandable guide device may be reusable.

In various embodiments described herein and otherwise within the scope of the disclosure, a method of performing an endoscopy may include inserting an endoscope into an instrument lumen of an expandable guide device. The instrument and expandable guide device may be together inserted into a patient. The endoscope and expandable guide device may be advanced through a body lumen to a treatment site within the patient. The expandable guide device may include a plurality of expandable members disposed circumferentially about the expandable guide device, wherein each member of the plurality of expandable members may be independently expandable radially between an unexpanded configuration, partially expanded configuration, and expanded configuration. The method may include expanding one or more members of the plurality of expandable members at the treatment site within the patient, such that the endoscope inserted through the instrument lumen of the expandable guide device is positionable radially, angularly, or both, relative to a central axis of the body lumen. The expandable members can be expanded within the patient, dilating or expanding into contact with the patient's body lumen, e.g., the duodenum, the intestines, or the like. Expanding the expandable member may facilitate holding the instrument still while using the instrument to examine other areas of the body. This can occur due to the outer diameter of the expandable member being dilated or pushing up in close contact to the body lumen, and inhibiting movement of the expandable guide device, as well as through selective expansion of expandable members to position the instrument at a particular radial position in the body lumen and/or at a particular angle of approach to the target tissue.

In various embodiments, multiple expandable guide devices can be used with a single instrument. FIG. 2B exemplifies this embodiment, showing a total of four devices used in tandem. Because the placement of the expandable guide device on the instrument can provide for variations in use, it may be beneficial to have more than one on the instrument. FIG. 2B shows various areas of the colon, some of which bend at significant angles. The expandable members of FIGS. 6A-B can assist in bending the instrument to move through these angled areas of the colon without harming the patient. In some embodiments, the use of multiple devices allows for each device to be independently manipulated, allowing them to be axially extended with respect to each other device. In this way, a device comprising all compliant expandable members may be used primarily on the distal end of the instrument for stabilization, but if, through extension through the body lumen, the instrument becomes looped, a device comprising at least one non-compliant expandable member may be used to resolve the loop.

Variations, modifications, and other implementations of the present disclosure in addition to the various embodiments described herein will occur to those of ordinary skill in the art. Accordingly, the present disclosure is to be defined not by the preceding illustrative description but instead by the following claims:

What is claimed is:

1. An expandable guide device, comprising:
a first body, comprising:
a proximal end, a distal end, the first body defining an instrument lumen extending therethrough along a longitudinal axis of the first body, and the instrument lumen configured to slidingly receive a first length of an instrument extendible therethrough; and
a plurality of expandable members disposed circumferentially about the first body, each member of the plurality of expandable members of the first body extending between a proximal end and a distal end along the longitudinal axis of the first body; and
wherein each member of the plurality of expandable members of the first body is independently expandable radially between an unexpanded configuration, partially expanded configuration, and expanded configuration, such that the first length of the instrument extendible through the instrument lumen of the first body is positionable radially, angularly, or both, relative to a central axis of a body lumen.

2. The device of claim 1, wherein two or more members of the plurality of expandable members of the first body are expandable, and further comprising a plurality of first elongate fluid delivery members, each first delivery member in fluid communication with a respective expandable member of the plurality of expandable members of the first body that are expandable.

3. The device of claim 1, further comprising a second body proximal to the first body, the second body comprising:
a proximal end, a distal end, the second body defining an instrument lumen extending therethrough along a longitudinal axis of the second body, and the instrument lumen configured to slidingly receive a second length of the instrument extendible therethrough; and
a plurality of expandable members disposed circumferentially about the second body, each member of the plurality of expandable members of the second body extending between a proximal end and a distal end along the longitudinal axis of the second body; and
wherein each member of the plurality of expandable members of the second body is independently expandable radially between an unexpanded configuration, partially expanded configuration, and expanded configuration, such that the second length of the instrument extendible through the instrument lumen of the second body is positionable radially, angularly, or both, relative to the central axis of the body lumen.

4. The device of claim 1, wherein two or more members of the plurality of expandable members of the second body are expandable, and further comprising one or more second elongate fluid delivery members, each second delivery member in fluid communication with a respective expandable member of the plurality of expandable members of the second body that are expandable.

5. The device of claim 1, further comprising:
a second body and a third body aligned axially with each other and the first body, each comprising a proximal end and a distal end, the second body and the third body each defining an instrument lumen extending therethrough along a longitudinal axis of the second body and the third body, the instrument lumen of the second body and the third body configured to slidingly receive a respective second length and third length of the instrument extendible therethrough;
a plurality of expandable members disposed circumferentially about the first body, the second body and the third body, the expandable members extending from the proximal end of the second body to the distal end of the first body, and from the distal end of the second body to the proximal end of the third body, along the longitudinal axis of the first, second and third body;
wherein the expandable members extending between the first and second bodies are configured to expand radially from the longitudinal axis of the first and second bodies when an axial length between the first and second bodies is decreased; and
wherein the expandable members extending between the second and third bodies are configured to expand radially from the longitudinal axis of the second and third bodies when an axial length between the second and third bodies is decreased.

6. The device of claim 5, wherein the first, second, or third bodies, or combinations thereof, are configured to be pushed or pulled along one or more of the longitudinal axes of the first, second and third bodies to selectively expand the expandable members between the first and second bodies, or the expandable members between the second and third bodies, or both.

7. The device of claim 1, wherein the plurality of expandable members of the first body comprises a plurality of turns forming a coil, and wherein the coil is disposed along the first body, the coil and the first body expandable radially when the distal end of the first body is fixed to the instrument and the proximal end of the first body is rotated about the longitudinal axis of the first body.

8. The device of claim 1, wherein the plurality of expandable members of the first body comprises a non-compliant material or a compliant material, or combinations thereof.

9. The device of claim 1, wherein the plurality of expandable members of the first body comprises two three, or four expandable members.

10. The device of claim 1, wherein the plurality of expandable members of the first body is spherical, oblong, bean-shaped, kidney-shaped, finger-shaped or ellipsoidal, or any combinations thereof.

11. The device of claim 4, wherein two or more members of the plurality of expandable members of the second body are expandable and non-compliant, and wherein the one or more second elongate fluid delivery members comprise a single elongate fluid delivery member in fluid communication with the two or more non-compliant expandable members.

12. The device of claim 1, wherein the plurality of expandable members of the first body comprises a shape memory metal or plastic material and is formed from round wires, flat wire, or woven mesh, or combinations thereof.

13. An endoscopic system, comprising:
an instrument; and
an expandable guide device slidably disposed about the instrument, comprising:
a first body, comprising:
a proximal end, a distal end, the first body defining an instrument lumen extending therethrough along a longitudinal axis of the first body, and the instrument lumen configured to slidingly receive a first length of the instrument extendible therethrough; and
a plurality of expandable members disposed circumferentially about the first body, each member of the plurality of expandable members extending between a proximal end and a distal end along the longitudinal axis of the first body; and
wherein each member of the plurality of expandable members of the first body is independently expandable radially between an unexpanded configuration, partially expanded configuration, and expanded configuration, such that the first length of the instrument extendible through the instrument lumen of the first body is positionable radially, angularly, or both, relative to a central axis of a body lumen.

14. The system of claim 13, wherein two or more members of the plurality of expandable members of the first body are expandable, and further comprising a plurality of first elongate fluid delivery members, each first delivery member in fluid communication with a respective expandable member of the plurality of expandable members of the first body that are expandable.

15. The system of claim 13, further comprising a second body proximal to the first body, the second body, comprising:
a proximal end, a distal end, the second body defining an instrument lumen extending therethrough along a longitudinal axis of the second body, and the instrument lumen configured to slidingly receive a second length of the instrument extendible therethrough; and
a plurality of expandable members disposed circumferentially about the second body, each member of the plurality of expandable members of the second body extending between a proximal end and a distal end along the longitudinal axis of the second body; and
wherein each member of the plurality of expandable members of the second body is independently expandable radially between an unexpanded configuration, partially expanded configuration, and expanded configuration, such that the second length of the instrument extendible through the instrument lumen of the second body is positionable radially, angularly, or both, relative to the central axis of the body lumen.

16. The system of claim 13, further comprising an over-sleeve slidably disposable about the expandable guide device.

17. The system of claim 13, wherein the plurality of expandable members of the first body comprises a compliant material or a non-compliant material.

18. The system of claim 13, wherein the plurality of expandable members of the first body comprises two, three, four, five, or more expandable members.

19. A method of performing an endoscopic procedure within a patient, comprising:
inserting an endoscope into an instrument lumen of an expandable guide device;
advancing the endoscope and the expandable guide device through a body lumen to a treatment site within the patient, the expandable guide device including a plurality of expandable members disposed circumferentially about the expandable guide device, wherein each member of the plurality of expandable members is independently expandable radially between an unexpanded configuration, partially expanded configuration, and expanded configuration; and
expanding one or more members of the plurality of expandable members at the treatment site within the patient, such that the endoscope inserted through the instrument lumen of the expandable guide device is positionable radially, angularly, or both, relative to a central axis of the body lumen.

20. The method of claim 19, wherein the plurality of expandable members comprises a non-compliant material or a compliant material, or combinations thereof.
